(19) United States
(12) Patent Application Publication
(10) Pub. No.: US 2025/0035644 A1
(43) Pub. Date: Jan. 30, 2025

(54) METHOD AND DEVICE FOR ANALYSING A SET OF SAMPLES OR A SURFACE

(71) Applicants: CENTRE NATIONAL DE LA RECHERCHE SCIENTIFIQUE, PARIS (FR); UNIVERSITE DE LORRAINE, NANCY (FR)

(72) Inventors: Pascal ROBERT, SEXEY-AUX-FORGES (FR); Jean CAUZID, VILLERS-LES-NANCY (FR)

(73) Assignees: CENTRE NATIONAL DE LA RECHERCHE SCIENTIFIQUE, PARIS (FR); UNIVERSITE DE LORRAINE, NANCY (FR)

(21) Appl. No.: 18/707,398

(22) PCT Filed: Oct. 20, 2022
(86) PCT No.: PCT/EP2022/079297

§ 371 Date: May 3, 2024

(30) Foreign Application Priority Data
Nov. 4, 2021 (FR) FR2111729

(51) Int. Cl. G01N 33/68

(52) U.S. Cl. CPC G01N 33/6851

(57) ABSTRACT

A multi-technique and pluri-decimetric chemical mapping method and device for studying a set of specimens, core samples or indeed a surface. The device includes a control computer, a control unit, a measurement instrument, at least one longitudinal rail extending in a direction referred to as longitudinal direction, a support mounted for translational movement on the longitudinal rail in the longitudinal direction, a plate mounted for translational movement on the support in a transvers direction and perpendicular to the longitudinal direction, a fixing intended for fixing the measurement instrument and mounted for translational movement on the plate in a direction referred to as vertical direction, mechanisms for bringing about the translational movement of, respectively, the support, the plate and the fixing, these mechanisms being governed by the control unit.

### FIELD

The present invention relates to a method and a device for multi-technique and pluri-decimetric chemical analysis for studying a set of samples or a surface.

### BACKGROUND

Devices are known for the physical, mineralogical and/or chemical characterisation of rocks, enabling several parameters such as density, magnetic susceptibility, mineralogy, etc. to be measured non-destructively. The measurements are taken on drill cores.

In particular, there is the device developed by Geotek. This system includes a bench and an analysis system. The bench includes an input conveyor on which the core to be analysed is placed, an analysis conveyor on which the core is transferred for analysis and an output conveyor on which the core is transferred after analysis. The core is then moved only along a so-called longitudinal axis X. The analysis system includes an analysis instrument and fastening elements. The analysis instrument is attached to the bench's analysis conveyor by means of the fastening elements. The analysis instrument scans the core during analysis along the axis X.

However, this device is of large overall size and limited in terms of the analysis instrument because the instrument can only move along the axis X. In addition, it is dedicated specifically to core analysis. It is also known, from Document FR 3 022 029 A1, a device for the spectroscopic analysis of drill core, especially drill core from oil development, mining or scientific operations, implementing a laser induced breakdown spectroscopy (LIBS) method. The device includes:
a measurement set comprising optical means for laser illumination, optical means for collecting the light from the plasma and an imaging camera;
at least one spectrometer connected to the optical collection means;
data processing means for processing the signals supplied by the at least one spectrometer and the images supplied by the camera;
a core support for supporting the core on a measuring table, and for holding the core in a predetermined position;

-means for carry out a relative movement between the measurement set and the core support.

However, this device is of large overall size and limited in terms of analysis instruments, since it is dedicated to Laser Induced Breakdown Spectroscopy (LIBS).

In addition, it is specifically dedicated to core analysis.

This is why the invention is directed to provide a multi-technique and pluri-decimetric chemical mapping device that is more ergonomic and offers protocol flexibility so as to enable the analysis of a plurality of media, that is, the analysis not only of cores but also of walls and samples.

### SUMMARY

To this end, one object of the invention is a multi-technique and pluri-decimetric chemical mapping device for studying a set of samples, cores or a surface, characterised in that said device includes a drive computer, a control unit, a measuring instrument, at least one first longitudinal rail extending in a so-called longitudinal direction, a support translatably mounted to said longitudinal rail in said longitudinal direction, a plate translatably mounted to said support in a so-called transverse direction perpendicular to said longitudinal direction, a fastener provided for fastening the measuring instrument, translatably mounted to said plate in a so-called vertical direction, means for actuating translation of the support, the plate and the fastener respectively, said means being slaved to the control unit.

Optional, complementary or alternative characteristics of the invention are set out below.

Advantageously, the measuring instrument can be selected from the list defined by infrared ray spectrometers, laser ablation spectroscopes, X-ray fluorescence spectrometers and RAMAN spectrometers.

In one embodiment, the device may also include a bench extending in the longitudinal direction and to which the at least one longitudinal rail is fastened.

Preferably, the bench can include a conveyor capable of moving the set of samples or cores in the so-called longitudinal direction.

Also preferably, the device may further include a compartmentalised tray provided for accommodating a set of samples and/or a crate provided for accommodating cores.

Even more preferably, the device may further include a set of stops and wedges for wedging and blocking said tray and/or said crate.

According to a certain embodiment, the device may include a second longitudinal rail and the support may include two slides translatably mounted to each of the longitudinal rails respectively.

Advantageously, the support can comprise at least one transverse rail to which the plate is translatably mounted in the so-called transverse direction.

According to another embodiment, an actuator can be fastened to the plate, the part of said actuator which is vertically translatably movable carrying the fastener of the measuring instrument.

Preferably, the means for actuating translation of the support, the plate and the fastener respectively may be electric motors.

Advantageously, the support is provided with means for fastening the support to a surface.

Advantageously, the device may include a point-line-plane system, including a fixed base, a movable base, three balls, two of which are housed in a trihedron and a V-shaped groove respectively, and three screws passing through the movable base and dedicated to securing one of the balls respectively.

The invention also relates to a multi-technique and pluri-decimetric chemical mapping method for studying a tray of samples or cores, using a multi-technique and pluri-decimetric chemical mapping device in accordance with one embodiment of the invention and including a bench, a conveyor as well as a compartmentalised tray for receiving a set of samples, and/or a crate for receiving cores, characterised in that the tray filled with a set of samples, or the crate filled with carrots, is positioned manually on the bench, and the tray or crate is then blocked when the bench includes a set of locking and clamping stops and wedges, the control unit then drives the movement of the measuring instrument, according to a programme run by the computer, in at least one of the three directions (X), (Y), (Z), so as to successively analyse the set of samples or the set of cores.

One object of the invention is also a multi-technique, pluri-decimetric chemical mapping method for studying a surface, using a multi-technique, pluri-decimetric chemical mapping device in accordance with one embodiment of the invention, wherein the support includes means for fastening it to a surface, said device further including a measuring instrument selected from the list defined by infrared spectrometers, laser ablation spectroscopes, X-ray fluorescence spectrometers and RAMAN spectrometers, characterised in that the support is fastened to the surface by means of the fasteners, and the control unit then drives the movement of the measuring instrument in at least one of the three directions (X), (Y) and (Z) in accordance with a programme run by the computer, so as to analyse a predefined zone of said surface.

### BRIEF DESCRIPTION OF THE FIGURES

Other advantages and features of the invention will become apparent upon reading the detailed description of non-limiting implementations and embodiments, and of the following appended drawings:

FIG. 1 This figure represents a schematic perspective view of a multi-technique, pluri-decimetric chemical mapping device in accordance with one embodiment of the invention.

FIG. 2 This figure represents a schematic top view of a multi-technique, pluri-decimetric chemical mapping device in accordance with one embodiment of the invention.

FIG. 3 This figure represents a schematic side view of a multi-technique, pluri-decimetric chemical mapping device in accordance with one embodiment of the invention.

FIG. 4 This figure represents a schematic front view of a multi-technique, pluri-decimetric chemical mapping device in accordance with one embodiment of the invention.

FIG. 5 This figure represents a detailed view of the mounting of an infrared spectrometer to a multi-technique, pluri-decimetric chemical mapping device in accordance with one embodiment of the invention.

FIG. 6 This figure represents a detailed view of the mounting of an X-ray fluorescence spectrometer to a multi-technique, pluri-decimetric chemical mapping device in accordance with one embodiment of the invention.

FIG. 7 This figure represents a detailed view of the mounting of a RAMAN spectrometer to a multi-technique, pluri-decimetric chemical mapping device in accordance with one embodiment of the invention.

FIG. 8 This figure represents a perspective view of a tray capable of accommodating a set of tubes containing samples that can be analysed on a multi-technique, pluri-decimetric chemical mapping device in accordance with one embodiment of the invention.

FIG. 9 This figure represents a perspective view of a Point-Line-Plane system implemented in a multi-technique, pluri-decimetric chemical mapping device in accordance with one embodiment of the invention.

FIG. 10 This figure represents a detailed view of a Point-Line-Plane system implemented in a multi-technique, pluri-decimetric chemical mapping device in accordance with one embodiment of the invention.

FIG. 11 This figure represents a detailed view of a set of wedging and blocking stops and wedges, implemented in a multi-technique and pluri-decimetric chemical mapping device in accordance with one embodiment of the invention.

FIG. 12 This figure represents another detailed view of a set of wedging and blocking stops and wedges, implemented in a multi-technique, pluri-decimetric chemical mapping device in accordance with one embodiment of the invention.

### DETAILED DESCRIPTION

As the embodiments described below are in no way limiting, it will be possible especially to consider alternatives of the invention comprising only a selection of characteristics described, isolated from the other characteristics described, even if this selection is isolated within a sentence comprising these other characteristics, if this selection of characteristics is sufficient to provide a technical advantage or to differentiate the invention from information in prior art.

This selection comprises at least one characteristic, preferably a functional characteristic without structural details, or with only a part of the structural details if this part alone is sufficient to provide a technical advantage or to differentiate the invention from the information in prior art.

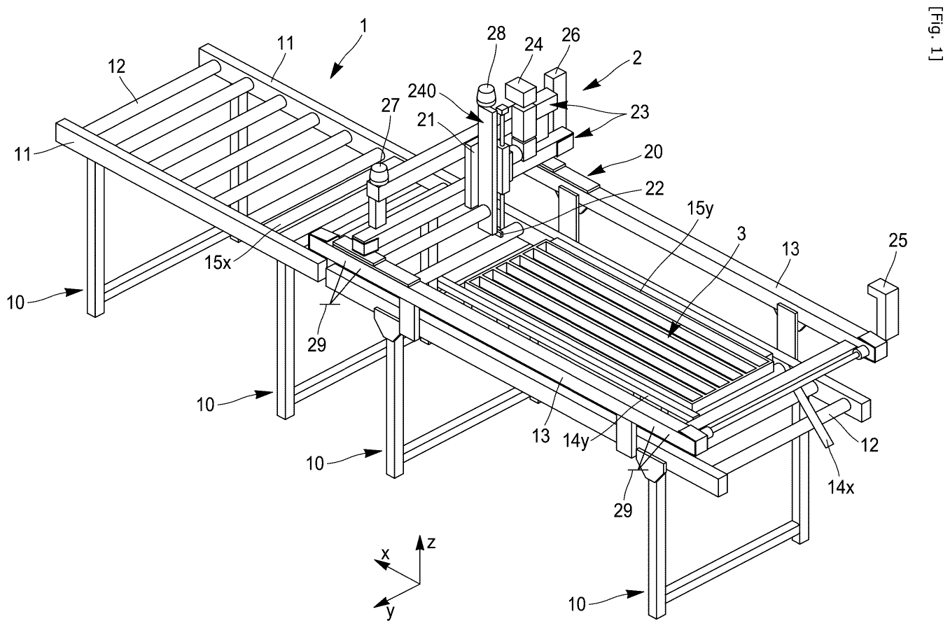

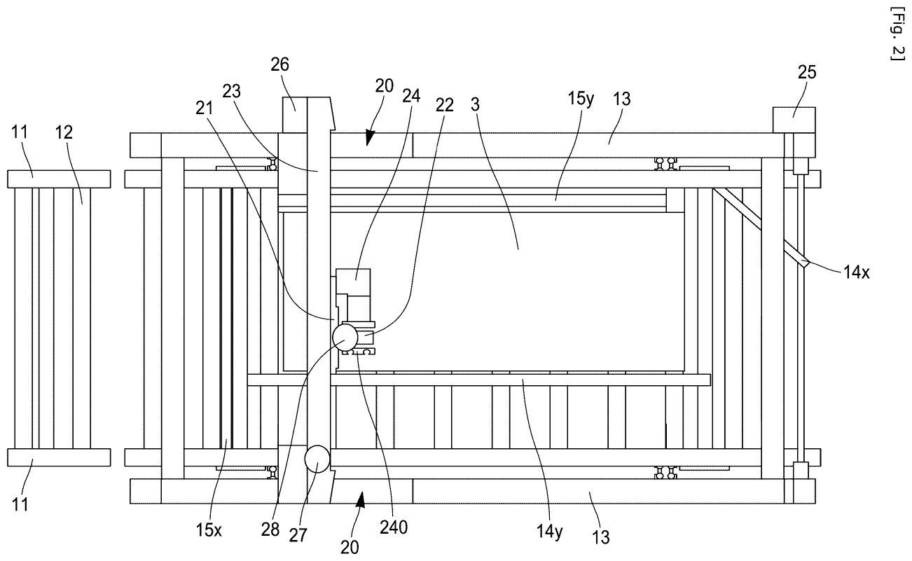

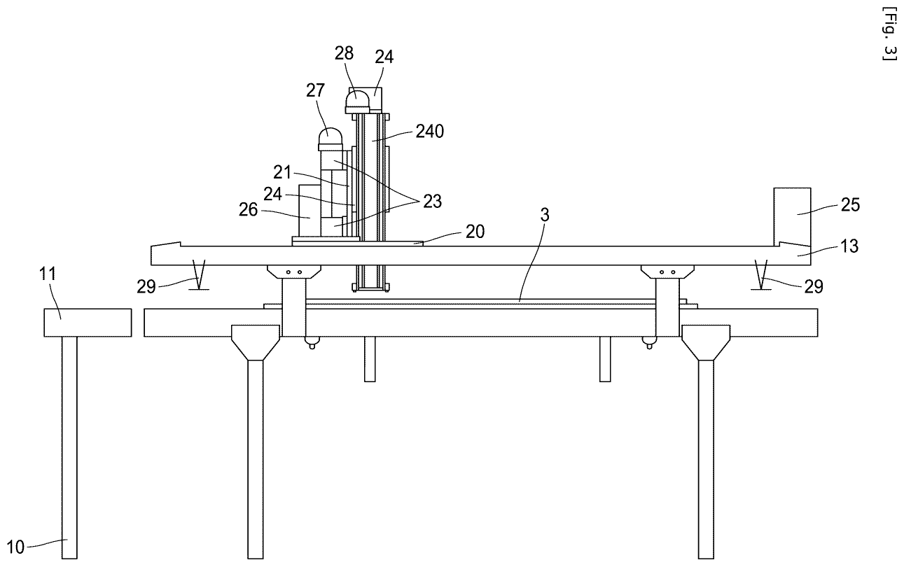

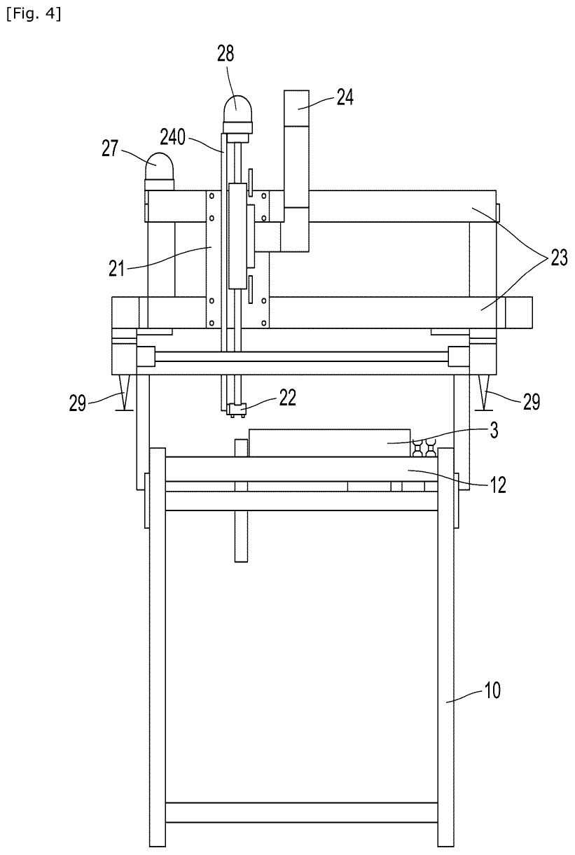

FIGS. 1 to 4 represent, from various points of view, a multi-technique, pluri-decimetric chemical mapping device for studying a set of samples, cores or a surface according to the principle of the invention.

The multi-technique and pluri-decimetric chemical mapping device includes at least one drive computer PC, a control unit ECU, a first rail 13 extending in a so-called longitudinal direction (X), a support 2 translatably mounted in said longitudinal direction to said rail 13, a plate 21 translatably mounted in a so-called transverse direction (Y) to said support, a fastener 22 for a measuring instrument, translatably mounted to said plate in a so-called vertical direction (Z).

Thus, the support 2 moves in the longitudinal direction (X), as do the plate 21 mounted to the support 2 and the fastener 22 mounted to the plate.

The plate 21 therefore moves in the longitudinal direction (X) as well as in the transverse direction (Y), as does the fastener 22 mounted to the plate.

The fastener 22 thus moves in the longitudinal direction (X), in the transverse direction (Y), as well as in the vertical direction (Z).

The device also includes means 24, 25, 26 for actuating translation of the support, the plate and the fastener respectively.

These means 24, 25, 26 are independently slaved to the control unit ECU, so that when the PC computer runs a programme, the control unit ECU drives the movement of the fastener (22) according to a specific programme. The programme is developed in LabVIEW and includes two parts:
the interface between the user and the device according to manual or automatic drive, and the control and display of the variables used in the programme. These can be switches, tables, text, etc.
A ‘source code’ diagram which shows the inputs and outputs of the interface and enables calculations to be made, external data to be retrieved, etc.

According to one embodiment represented in FIGS. 1 to 4, the multi-technique and pluri-decimetric chemical mapping device can advantageously include a bench 1 on which the support 2 is made to rest. In this case, at least one or two longitudinal rails 13 is/are fastened to the bench and the support 2 includes two slides 20 each translatably mounted to one of the two longitudinal rails 13 integral with the bench 1.

The support 2 advantageously comprises at least one transverse rail 23 to which the plate 21 is translatably mounted in the transverse direction (Y).

According to the embodiment represented in FIGS. 1 to 4, the bench 1 advantageously includes a conveyor 11 capable of moving material in the so-called longitudinal direction X. This conveyor is provided with a plurality of fixed rollers 12.

Advantageously, the bench can include a set of stops and wedges for wedging and blocking trays of samples or crates of cores to be analysed.

Thus, as represented in FIGS. 11 and 12, this set comprises a clamping stop 14y in the transverse direction (Y), a clamping stop 14x in the longitudinal direction (X), a blocking stop 15x in the longitudinal direction (X) and a clamping wedge 15y. The stops 14x, 15x, 14y are actuated by means of jacks 140x, 150x, 140y.

An actuator 24 is advantageously fastened to the plate 21, the movable part 240 of said actuator carrying the fastener 22 of the measuring instrument 4.

Preferably, the means 24, 25, 26 for actuating translation of the support, the plate and the fastener respectively are electric motors.

Advantageously, the device includes feet 10 on which the bench 1 rests.

Equally advantageously, the support is provided with means 29 for fixing it to a surface.

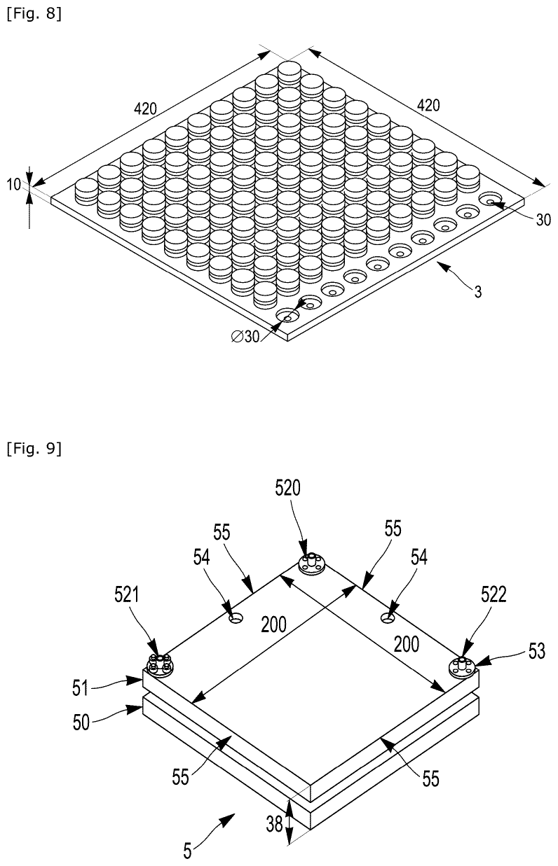

As represented in FIG. 8, the device further includes a compartmentalised tray 3 so as to accommodate a set of samples.

This tray can be a moulded plastic plate which includes, on one of its faces, recesses 30 provided at regular intervals and capable of accommodating tubes filled with samples (generally powders). As the geometry of the analysis tubes is identical for all, there is no difference in height between the tubes and therefore no adjustment to be made (no tilt to be modified).

According to one particular embodiment, the tray 3 more precisely has 100 slots distributed in 10 rows and 10 columns. These slots are sized to receive small PVC tubes or boxes filled with powder of the materials to be analysed. These boxes are closed with a plastic film that does not interfere with X-ray fluorescence analysis. This system can be used on a multi-technique, pluri-decimetric chemical mapping device in accordance with one embodiment of the invention.

The device may also comprise a crate capable of accommodating cores.

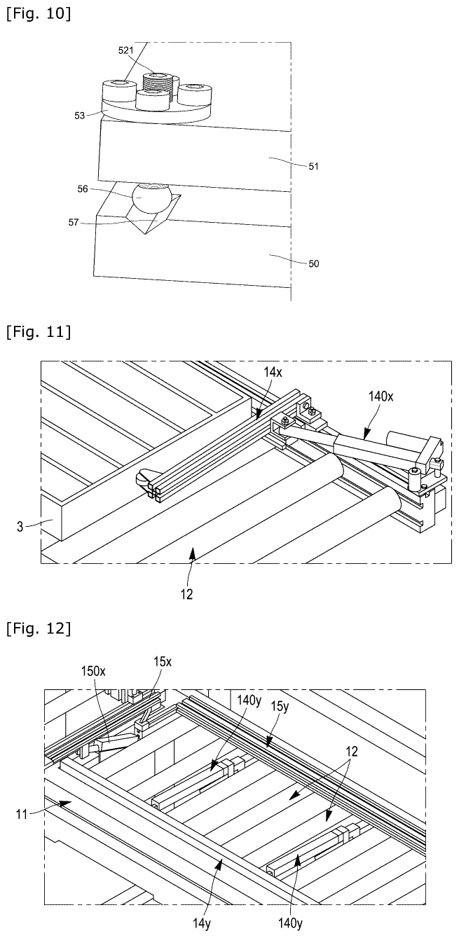

As represented in FIGS. 9 and 10, the device advantageously includes a Point-Line-Plane 5 system.

This device enables the surface to be analysed and the horizontal plane of the bench to be made parallel. Thus, the distance between a point on the plane of the surface to be analysed and an instrument 4 remains constant.

The Point-Line-Plane system consists of a fixed base 50, a movable base 51 delimited by four edges 55, three balls 56, two of which are housed in a trihedron 57 and the third in a V-shaped groove, and three screws 520, 521, 522, each passing through an insert 53 as well as the movable base and respectively dedicated to securing one of the balls. The position of the fixed base 50 relative to the movable base 51 is adjusted by means of two tension springs 54 and the three screws 520, 521, 522.

Advantageously, the device includes a first indicator light 27 integral with the support 2 as well as a second indicator light 28 integral with the plate 21.

The first indicator light is activated to signal the movements of the analysis instruments. The second indicator light is activated to signal that an analysis instrument is in operation, with a potential danger. It is implemented especially during:
XRF analysis, due to the emission of X-rays,
Raman analysis, due to the emission of a class 3 laser beam,
LIBS analysis, due to class 4 laser shots.

Advantageously, in the fields of application relating to archaeology or geology, the device includes a measuring instrument selected from the list defined by infrared spectrometers, laser ablation spectroscopes, X-ray fluorescence spectrometers and RAMAN spectrometers.

Infrared spectroscopy is a class of spectroscopy that deals with the infrared region of the electromagnetic spectrum. It covers a wide range of techniques, the most common being a type of absorption spectroscopy. As with all spectroscopic techniques, it can be utilised to identify compounds or to determine the composition of a sample. As the acquisition instruments are miniaturised, they can be transported, even for outdoor use. With the development of computing filtering and result processing technologies, samples in a solution can now be measured precisely (water has a high absorbance at wavelengths of interest, making an unprocessed spectrum uninterpretable). As some instruments have their own databases, identification can thus also be automated.

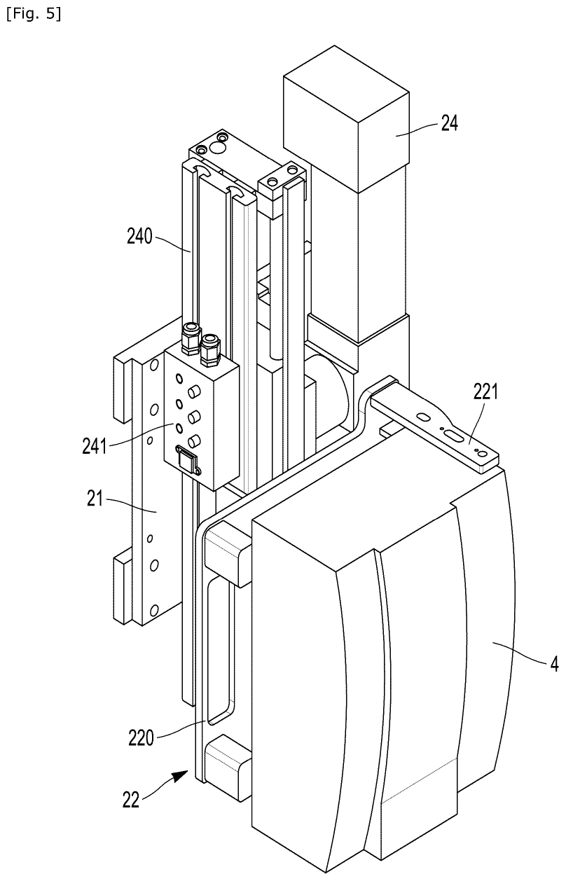

FIG. 5 represents in detail the mounting of an IR spectrometer 4 to the device according to the invention. More particularly, the IR spectrometer 4 is fastened to a backing 220 and held in place by means of a lug 221, thus constituting the fastener 22. The backing 220 is itself mounted to the movable part 240 of the actuator 24, which is itself mounted to the plate 21. A connection box 241 enables the various instruments to be electrically connected to the drive PC. This box includes a USB socket and three specific sockets adapted to the various instruments. The actuator 24 is driven by the ECU.

Laser induced plasma spectrometry is a very minimally destructive analytical technique (micron surface ablation), the principle of which is based on the use of non-ionising monochromatic radiation.

Sample analysis have five steps:
1—A high-energy pulsed laser is focused on the sample. The high temperature of the laser on the sample results in the ablation of a small volume of material in a plasma. The plasma contains excited atoms and ions of the sample. The environment is controlled: air, argon, helium.
2—When the plasma begins to cool, the electrons of the excited atoms and ions come back into their initial states. As they return to their ground states, light (of specific wavelengths) is emitted by the plasma and collected by the spectrometer.
3—The spectrometer separates all the wavelengths using high-resolution dispersive optics and then detects them using a charge-coupled device (CCD) containing a very large number of pixels aligned in a photodiode array.
4—Each pixel (after wavelength calibration) corresponds to a precise, known wavelength. The intensity collected by each pixel is proportional to the amount of photons collected. The wavelength (axis X) and the intensity (axis Y) are used to display a histogram (spectrum) characteristic of an ablated point (therefore of the sample if it is homogeneous).
5—Thus, if the pixel under consideration corresponds to the wavelength of a de-excitation characteristic of an electronic transition of an element, an appropriate calibration can calculate the mass concentration of this element in the matrix. By processing the entire spectrum, the concentration of all the elements present, from hydrogen to uranium (if they have been calibrated) can be deduced.

X-ray fluorescence spectrometry is a chemical analysis technique that uses a physical property of the material, the fluorescence of X-rays.

When the material is bombarded with X-rays, it re-emits energy in the form of X-rays, among other things, this is X-ray fluorescence, or secondary X-ray emission.

The spectrum of X-rays emitted by the material is characteristic of the composition of the sample, by analysing this spectrum, the elemental composition, that is, the mass concentrations of elements can be deduced.

The spectrum can be analysed in two ways:
either by wavelength dispersive X-ray fluorescence spectrometry (WD-XRF) analysis;
or by energy dispersive X-ray fluorescence spectrometry (ED-XRF) analysis.

FIG. 6 illustrates a fastener 22 of an X-ray fluorescence spectrometer to a device according to the invention. The head of the X-ray fluorescence spectrometer is fastened to a support 222′ which is itself mounted to a plate 221′. Four threaded rods 223′ are screwed to this plate. The rods pass through two horizontal bars 220′ which are integral with the movable part 240 of the jack Z. Springs inserted between the support plate and the horizontal bars cushion the contact between the spectrometer head and the part to be analysed. This contact has to be soft (to avoid inducing mechanical stress on the instrument and the sample) and firm (to be able to activate the contact that enables the instrument to send its X-ray flow). This is a safety feature because if there is no contact, there is no flow. A double nut system at the top of the threaded rods is used to adjust the stiffness of the contact between the plate and the sample.

Raman spectroscopy (or Raman spectrometry) and Raman microspectroscopy s for observing and characterising the molecular composition and external structure of a material, utilising the physical phenomenon whereby a medium slightly alters the frequency of the light circulating therein. This frequency shift, known as the Raman effect, corresponds to an exchange of energy between the light beam and the medium, and provides information about the substrate itself. Raman spectroscopy consists in sending monochromatic light onto the sample and analysing the scattered light. The information obtained by measuring and analysing this shift enables certain properties of the medium to be determined, by spectroscopy.

Coherent Raman scattering does not use an observation of light scattered spontaneously during molecular collisions, but the coherent amplification of a second beam of different frequency and temporally incoherent with the exciting beam.

This technique complements infrared spectroscopy. Both enable the study of the vibrational modes of a molecule, but the rules for selecting the two spectroscopies may differ depending on molecular symmetry. In molecules with a centre of symmetry, no vibrational mode can be observed at a time using both spectroscopies. Some modes are active only in the Raman and others only in the infrared.

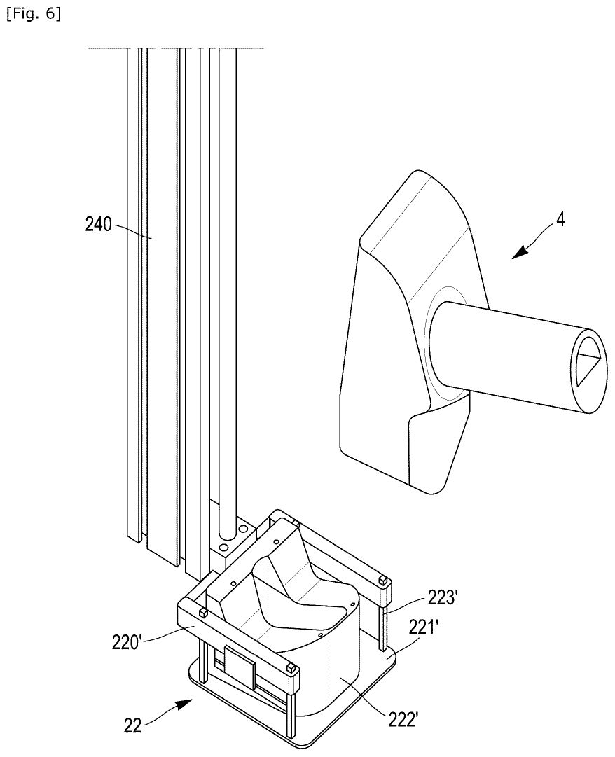

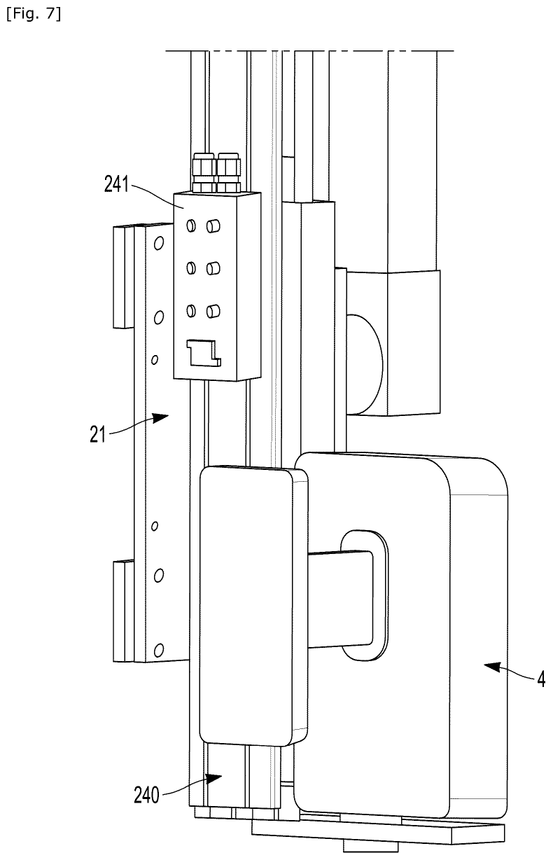

FIG. 7 illustrates mounting of a RAMAN spectrometer to a device according to the invention. The round head of the spectrometer is blocked in the cylindrical hole of a plate which is fastened to the base of the movable part 240 of the actuator Z. The spectrometer is held in balance by two nylon screws that tighten its head to the plate. The spectrometer is connected to the USB socket of the box 241. Its power supply is fastened to the ‘instrument power supply support’.

Such a device according to the invention makes it possible to carry out the analysis directly in a mine, on an archaeological site or even a geological site without needing to drill the support to extract a core.

The device enables analysis both horizontally and inclined to the vertical, and enables analysis both on soils and walls.

To do this, the support is fastened to the surface to be analysed by means of the fasteners 29, then the control unit drives the movement of the measuring instrument according to a programme run by the computer, in at least one of the three directions (X), (Y), (Z), so as to analyse a predefined zone of said surface (soil or wall).

In the case of multi-technique and pluri-decimetric chemical mapping of a tray of samples or cores, the tray 3 filled with a set of samples, or the crate filled with cores, is positioned manually on the bench, then the tray or crate is blocked when the bench includes a set of stops and wedges 14x, 14y, 15x, 15y. Electrical control is performed by means of a control box equipped with push buttons. This operation is not automated. To automate this operation, a sequence of actions would have to be carried out (1—blocking stop X, 2—clamping Y, 3—clamping X), the electrical currents in the jacks would have to be measured and the movement of each jack would have to be stopped when its current rises above a threshold to be defined. The increase in current corresponds to a mechanical resistance generated by the clamping X and Y. For the stop X, the system design does not require current measurement.

The control unit then drives the movement of the measuring instrument in at least one of the three directions (X), (Y), (Z), according to a programme run by the computer, so as to successively analyse all the samples or all the cores.

In addition, the analysis instrument may be variable as a function of the mapping to be carried out: infrared, LIBS, Raman, X-ray fluorescence, high-resolution photography. It is also possible to combine several analysis instruments so as to specify the chemical composition of the zones analysed.

The device according to the invention enables minerals to be mapped with chemical and optical characterisation instruments (infrared, laser ablation spectroscopy (LIBS), Raman, X-ray fluorescence, high-resolution photography).

The use of a combination of instruments enables the chemical composition of the zones analysed to be specified.

The nature of the mineral elements can be of any type: geological samples, archaeological samples (mosaics, paintings, for example).

The device according to the invention can be used to work on planar or quasi-planar samples of centimetre to metre size, such as geological cores.

The device can be positioned horizontally or inclined to the vertical. It enables analyses to be carried out both on soils and on walls (mine faces, for example).

The device according to the invention eliminates the tedious and repetitive nature of manual analyses. For example, for X-ray fluorescence analyses, analysis times can reach several minutes for a same point without moving.

The precision of the positioning of the instruments and their movements ensures that the analyses carried out by the different instruments are superimposed, so that the chemical composition of the sampled points can be accurately specified.

The system can be transported in a van or any other vehicle weighing less than 3.5 tonnes.

It should be noted that the various characteristics, forms, alternatives and embodiments of the invention may be associated with one another in various combinations insofar as they are not incompatible or mutually exclusive.

1-12. (canceled)

13. A multi-technique and pluri-decimetric chemical mapping device for studying a set of samples, cores or a surface, wherein said device includes a PC drive computer, a control unit ECU, a measuring instrument, at least one longitudinal rail extending in a so-called longitudinal direction, a support translatably mounted to said longitudinal rail in said longitudinal direction, a plate translatably mounted to said support in a so-called transverse direction perpendicular to said longitudinal direction, a fastener provided for fastening the measuring instrument and translatably mounted to said plate in a so-called vertical direction, means for actuating translation of the support, the plate and the fastener respectively, said means for actuating translation being slaved to the control unit ECU.

14. The multi-technique and pluri-decimetric chemical mapping device according to claim 13, wherein the measuring instrument is selected from the list defined by infrared ray spectrometers, laser ablation spectroscopes, X-ray fluorescence spectrometers and RAMAN spectrometers.

15. The multi-technique and pluri-decimetric chemical mapping device according to claim 13, further comprising a bench extending along the longitudinal direction and to which the at least one longitudinal rail is fastened.

16. The multi-technique and pluri-decimetric chemical mapping device according to claim 15, wherein the bench includes a conveyor capable of moving the set of samples or cores in the so-called longitudinal direction.

17. The multi-technique and pluri-decimetric chemical mapping device according to claim 16, further comprising a compartmentalised tray provided for accommodating a set of samples, and/or a crate provided for accommodating cores.

18. The multi-technique and pluri-decimetric chemical mapping device according to claim 17, wherein the bench includes a set of stops and wedges for blocking and wedging said tray and/or said crate.

19. The multi-technique and pluri-decimetric chemical mapping device according to claim 13, wherein the support comprises at least one transverse rail to which the plate is translatably mounted in the so-called transverse direction.

20. The multi-technique and pluri-decimetric chemical mapping device according to claim 13, wherein the means for actuating translation of the fastener, the support and the plate respectively are electric motors.

21. The multi-technique and pluri-decimetric chemical mapping device according to claim 13, wherein the support is provided with means for fastening said support to a surface.

22. The multi-technique and pluri-decimetric chemical mapping device according to claim 13, further comprising a Point-Line-Plane system, including a fixed base, a movable base, three balls, two of which are housed respectively in a trihedron and a V-shaped groove, three screws passing through the movable base and dedicated respectively to securing one of the balls.

23. A multi-technique and pluri-decimetric chemical mapping method for studying a tray of samples or cores, using a device in accordance with claim 18, wherein the tray filled with a set of samples, or the crate filled with cores, is positioned manually on the bench, the tray or the crate is then blocked when the bench includes a set of blocking and clamping stops and wedges, then the control unit ECU drives the movement of the measuring instrument in at least one of the three directions, according to a programme run by the PC computer, so as to successively analyse the set of samples or the set of cores.

24. A multi-technique and pluri-decimetric chemical mapping method for studying a surface, using a device in accordance with claim 21, said device further including a measuring instrument selected from the list defined by infrared ray spectrometers, laser ablation spectroscopes, X-ray fluorescence spectrometers, RAMAN spectrometers, characterised in that the support is fastened to the surface by the fasteners, and then the control unit ECU drives the movement of the measuring instrument in at least one of the three directions, according to a programme run by the PC computer, so as to analyse a predefined zone of said surface.
